(19) United States
(12) Patent Application Publication
(10) Pub. No.: US 2024/0000824 A1
(43) Pub. Date: Jan. 4, 2024

(54) OLIGONUCLEOTIDES CONTAINING 2'-DEOXY-2'FLUORO-BETA-D-ARABINOSE NUCLEIC ACID (2'-FANA) FOR TREATMENT AND DIAGNOSIS OF RETROVIRAL DISEASES

(71) Applicants: CITY OF HOPE, Duarte, CA (US); AUM LIFE TECH, INC., Philadelphia, PA (US); THE ROYAL INSTITUTION FOR THE ADVANCEMENT OF LEARNING/MCGILL UNIVERSITY, Montreal (CA)

(72) Inventors: John J. ROSSI, Azusa, CA (US); Masad J. DAMHA, Montreal (CA); Veenu AISHWARYA, Philadephia, PA (US); Mayumi TAKAHASHI, Pasadena, CA (US); Haitang LI, Duarte, CA (US)

(73) Assignees: CITY OF HOPE, Duarte, CA (US); AUM LIFE TECH, INC., Philadelphia, PA (US); THE ROYAL INSTITUTION FOR THE ADVANCEMENT OF LEARNING/MCGILL UNIVERSITY, Montreal (CA)

(21) Appl. No.: 18/344,507

(22) Filed: Jun. 29, 2023

Related U.S. Application Data
(62) Division of application No. 16/335,663, filed on Mar. 21, 2019, now abandoned.
(60) Provisional application No. 62/399,101, filed on Sep. 23, 2016.

(51) Int. Cl. A61K 31/7115; A61K 31/7125; C12N 15/113

(52) U.S. Cl. CPC A61K 31/7115; A61K 31/7125; C12N 15/1132; C12N 2310/11; C12N 2310/315; C12N 2310/323; C12N 2310/341; C12N 2310/346

(57) ABSTRACT

The disclosure relates to synthetic oligonucleotides that bind at least a portion of a dimerization initiation site (DIS) of a retrovirus genomic ribonucleic acid (RNA) molecule. In some aspects, the synthetic oligonucleotides include a 2′-deoxy-2′-fluoroarabinonucleotide (2′-FANA)-modified nucleotide sequence. In some embodiments, the 2′-FANA-modified nucleotide sequence inhibits dimerization of retroviral genomes (e.g., an HIV genome). Other embodiments include methods of inhibiting expression of a retrovirus using the synthetic oligonucleotide, and methods of treating or preventing a retroviral infection.

### PRIORITY CLAIM

This application is a divisional of U.S. application Ser. No. 16/335,663, filed Mar. 21, 2019, which is a U.S. national phase application of International Application No. PCT/US2017/053127, filed Sep. 23, 2017, which claims the benefit of U.S. Provisional Application No. 62/399,101, filed Sep. 23, 2016, the subject matter of which are incorporated by reference as if fully set forth herein.

### STATEMENT REGARDING FEDERALLY SPONSORED RESEARCH

This invention was made with government support under AI029329 and R01HL074704 awarded by the National Institutes of Health. The government has certain rights in the invention.

### SEQUENCE LISTING

This application contains a ST.26 compliant Sequence Listing, which was submitted in XML format via Patent Center, and is hereby incorporated by reference in its entirety. The XML copy, created on Jun. 29, 2023, is named 0544358152US02.xml and is 64,000 bytes in size.

### FIELD

The disclosure relates to synthetic oligonucleotides and uses thereof, particularly for the treatment of viral diseases, such as human immunodeficiency virus (HIV).

### BACKGROUND

Viruses cause many common diseases including, smallpox, the common cold, shingles, herpes, human immunodeficiency virus (HIV) infections, and some types of cancers, to name a few. Virion particles, the viral form prior to cell entry, are made up of genetic material (e.g., DNA or RNA), a protein coat, and a lipid envelope and use receptors and co-receptors to enter a cell. HIV, for example, targets CD4⁺ immune cells such as T-helper cells, macrophages and dendritic cells. Upon host cell entry, viruses replicate to reproduce more viral proteins and genetic material. Single stranded RNA retroviruses, like HIV, use reverse transcriptase to transcribe into DNA and then are integrated into the host cell. After integration, cells create more virus, often different viral strains.

Various treatment methods, such as use of anti-retroviral therapy (ART), often help to increase lifespan and reduce risks of transmission of viruses; however, despite decades of research, there are no cures for several viral diseases, including HIV. Left untreated, HIV progresses into acquired immune deficiency syndrome (AIDS). Since 2000, approximately 38 million people have become infected with HIV and approximately 25 million people have died as a result of secondary illnesses caused by AIDS.

Single-stranded synthetic oligonucleotides, referred to as antisense oligonucleotides (ASOs or AONs), recognize target RNAs and cause post-transcriptional gene silencing. The mechanisms are believed to be RNase H-induced cleavage of target RNA, steric hindrance of the translation machinery or prevention of RNA-RNA or RNA-protein interactions. Vickers et al., (2014) PLoS One 9, e108625; Lima et al., (2007) Mol. Pharmacol. 71:83-91; Lima et al., (2007) Mol. Pharmacol. 71:73-82. While AONs offer promising solutions for variety of human diseases in preclinical studies, and many of these are currently in clinical studies, a number of challenges still hamper their translation from the bench to the bedside. The most significant challenges include target accessibility, off target effects, poor extracellular and intracellular stability and effective delivery into target cells. Chan et al., (2006) Clin Exp Pharmacol Physiol, 33:533-540; Geary et al., (2015) Adv Drug Deliv Rev, 87:46-51; Gogtay et al., (2016) Br J Clin Pharmacol, 28:3625-3635.

There remains a need for improved AON chemistries and designs for use as treatments for viral diseases.

### SUMMARY

Synthetic oligonucleotides comprising a 2′-deoxy-2′-fluoroarabinonucleotide (2′-FANA)-modified nucleotide sequence are disclosed herein. In certain embodiments, the synthetic oligonucleotides bind at least a portion of a dimerization initiation site (DIS) of a retrovirus genomic ribonucleic acid (RNA) molecule. In some embodiments, the 2′-FANA-modified nucleotide sequence inhibits dimerization of retroviral genomes (e.g., an HIV genome).

Other embodiments include methods of inhibiting expression of a retrovirus. In certain embodiments, these methods include a step of delivering a synthetic oligonucleotide to a cell infected with a retrovirus. The synthetic oligonucleotide may comprise a 2′-deoxy-2′-fluoroarabinonucleotide (2′-FANA)-modified nucleotide sequence as described above. In certain embodiments, the delivery is accomplished by a gymnotic delivery method. The methods may be used to inhibit expression of the retrovirus in a population of cultured cells (i.e., in vitro) or in a population of cells found in a subject (i.e., in vivo).

In certain embodiments, the synthetic oligonucleotides described above are part of a composition. The composition may be used in a method of treating or preventing a retroviral infection in a subject, according to certain embodiments. Such methods may include a step of administering an effective amount of the composition to the subject.

### BRIEF DESCRIPTION OF THE DRAWINGS

FIG. 1 shows a schematic of the HIV-1 dimerization pathway. The 5′-untranslated region (UTR) of the HIV-1 RNA genome contains replication signals that are required in various steps in the replication cycle, including dimerization initiation site (DIS). Dimerization is initiated by conformation change of 5′-UTR from long-distance interaction (LDI) to branched multiple hairpin (BMH). This allows the DIS loop to intermolecular base pair between two RNA genomes, forming a kissing-loop (KL) dimer, followed by subsequent RNA packaging. (TAR: trans-acting response element, polyA: polyadenylation signal, PBS: primer binding site).

FIG. 2 is a schematic of an anti-HIV strategy for promoting inhibition of dimerization according to one embodiment of the present disclosure. As shown, an antisense oligonucleotide (AON) having 2′-FANA sequences on either side of a nucleotide gap sequence, with the sugar backbone of the individual bases linked together by phosphorothioate (PS) linkages can bind to the kissing loop region of HIV-1, which prevents dimerization and subsequent packaging to ultimately inhibit viral expression.

FIG. 3A shows representative confocal images of peripheral blood mononuclear cells (PBMCs) following gymnotic delivery of various concentrations of 2′-FANA modified antisense oligonucleotide (AONs) of two different nucleotide lengths.

FIG. 3B shows representative confocal images of a 6 hour time course internalization study of a 21-nucleotide length 2′-FANA AON.

FIG. 4A demonstrates cell uptake of Cy3-labeled 2′-FANA AONs into cells after incubation for 4 hours. Arrows point to Cy3 signaling in cytoplasm of cells.

FIG. 4B shows representative confocal images of CEM cells (a human leukemia T-cell cell line) following gymnotic delivery of various concentrations of 2′-FANA modified AONs of two different nucleotide lengths.

FIG. 5A is a schematic of a HIV challenge schedule according to one embodiment of the present disclosure.

FIG. 5B is a schematic of several 2′-FANA AON sequences according to an embodiment of the present disclosure.

FIG. 5C shows the inhibitory effects of HIV-1 expression by two 2′-FANA AONs (i.e., DIS-6 and DIS-7) over time in HIV infected PBMCs as compared to control (i.e., “Cells only”) based on expression of p24.

FIG. 5D shows the inhibitory effects of HIV-1 expression by two 2′-FANA AONs (i.e., DIS-6 and DIS-7) in HIV infected PBMCs as compared to a control ASO containing all nucleotides (i.e., “Unmodified”) and a control containing no ASO (i.e., “Cells only”) based on expression of p24.

FIG. 5E demonstrates AONs DIS-6 and DIS-7 do not elicit an immune response as determined by interferon-alpha (IFN-alpha) expression levels as compared to a positive control CpG 2395.

FIG. 5F demonstrates AONs DIS-6 and DIS-7 do not elicit an immune response as determined by interleukin-6 (IL-6) expression levels as compared to a positive control CpG 2395.

FIG. 6 shows results of in vitro dimerization assays and demonstrates that 2′-FANA AONs inhibit target RNA dimerization.

FIG. 7 shows results of an in vitro cleavage assay and demonstrates that 2′-FANA AONs mediate human RNase H1 cleavage of target RNA.

FIG. 8 is a table of exemplary modified synthetic oligonucleotides in accordance with the embodiments described herein. Bold and underlined nucleotides represent sugar-modified or 2′-FANA-modified nucleotides.

### DETAILED DESCRIPTION

It is to be understood that this disclosure is not limited to particular embodiments described, as such may, of course, vary. It is also to be understood that the terminology used herein is for the purpose of describing particular embodiments only, and is not intended to be limiting, since the scope of this disclosure will be limited only by the appended claims.

The detailed description of the disclosure is divided into various sections only for the reader's convenience and disclosure found in any section may be combined with that in another section. Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated by reference to disclose and describe the methods and/or materials in connection with which the publications are cited.

All numerical designations, e.g., pH, temperature, time, concentration, and molecular weight, including ranges, are approximations which are varied (+) or (−) by increments of 0.1 or 1.0, where appropriate. It is to be understood, although not always explicitly stated that all numerical designations are preceded by the term “about.” It also is to be understood, although not always explicitly stated, that the reagents described herein are merely exemplary and that equivalents of such are known in the art.

It must be noted that as used herein and in the appended claims, the singular forms “a”, “an”, and “the” include plural referents unless the context clearly dictates otherwise. Thus, for example, reference to “oligonucleotide” includes a plurality of oligonucleotides.

### Definitions

As used herein the following terms have the following meanings.

The term “about” when used before a numerical designation, e.g., temperature, time, amount, concentration, and such other, including a range, indicates approximations which may vary by (+) or (−) 20%, 10%, 5% or 1%.

Also as used herein, “and/or” refers to and encompasses any and all possible combinations of one or more of the associated listed items, as well as the lack of combinations when interpreted in the alternative (“or”).

“Comprising” or “comprises” is intended to mean that the compositions, for example synthetic oligonucleotides, and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this invention.

The terms “administering,” “administer” and the like refer to introducing an agent (e.g., an AON) into a patient. Typically, an effective amount is administered, which amount can be determined by the treating physician or the like. Any route of administration, such as topical, subcutaneous, peritoneal, intravenous, intraarterial, inhalation, vaginal, rectal, nasal, buccal, introduction into the cerebrospinal fluid, or instillation into body compartments can be used. The terms and phrases “administering” and “administration of,” when used in connection with a compound or pharmaceutical composition (and grammatical equivalents) refer both to direct administration, which may be administration to a patient by a medical professional or by self-administration by the patient, and/or to indirect administration, which may be the act of prescribing a drug. For example, a physician who instructs a patient to self-administer an agent (e.g., an AON) and/or provides a patient with a prescription for a drug is administering the agent to the patient. “Periodic administration” or “periodically administering” refers to multiple treatments that occur on a daily, weekly, or a monthly basis. Periodic administration may also refer to administration of an agent one, two, three or more time(s) per day.

The term “antiretroviral” in reference to a drug therapy (antiretroviral therapy (“ART”)) refers to administration of one or more antiretroviral drugs to inhibit replication of HIV. Typically, ART involves the administration of one antiretroviral agent (or, commonly, a cocktail of antiretrovirals) such as nucleoside reverse transcriptase inhibitor(s) (e.g., zidovudine, AZT, lamivudine (3TC) and abacavir), non-nucleoside reverse transcriptase inhibitor (e.g., nevirapine and efavirenz), and protease inhibitor(s) (e.g., indinavir, ritonavir, and lopinavir).

As used herein the term “arabinucleotide” refers to a nucleotide comprising an arabinofuranose sugar.

As used herein the term “complementary” refers to a nucleic acid sequence that is either fully or partially complementary to its target nucleic acid sequence. An oligonucleotide need not be 100% complementary to that of its target molecule to bind and specifically hybridize to the target. Thus, the nucleotide sequences described herein can be fully complementary (e.g., Watson-Crick pairing) to the target molecule or can have partial complementarity to the target molecule, for example, wobble base pairing (e.g., guanine-uracil, hypoxanthine-uracil, hypoxanthine-adenine, and hypoxanthine-uracil). In some aspects, the nucleotide sequences described herein may have at least 70% sequence complementarity to its target sequence, at least 80% sequence complementarity to its target sequence, at least 90% sequence complementarity to its target sequence, at least 95% sequence complementarity to its target sequence, at least 99% sequence complementarity to its target sequence, or may have 100% sequence complementarity to its target sequence.

As used herein the term “equivalents thereof” refers to an agent (e.g., AON and anti-retroviral drug) with the same or similar function and/or the same or similar ingredients. For example, an equivalent nucleic acid is a nucleic acid having a nucleotide sequence having a certain degree of homology with the nucleotide sequence of the nucleic acid or complement thereof. A homolog of a double stranded nucleic acid is intended to include nucleic acids having a nucleotide sequence which has a certain degree of homology with or with the complement thereof. In one aspect, homologs of nucleic acids are capable of hybridizing to the nucleic acid or complement thereof.

An “effective amount” is an amount of an agent or compound (e.g., AON and anti-retroviral drug) sufficient to effect beneficial or desired results. An effective amount can be in one or more administrations, applications or dosages. Determination of these parameters is well within the skill of the art. These considerations, as well as effective formulations and administration procedures are well known in the art and are described in standard textbooks.

“Identity” refers to sequence similarity between two nucleic acid molecules. Homology can be determined by comparing a position in each sequence which may be aligned for purposes of comparison. When a position in the compared sequence is occupied by the same base, then the molecules are homologous at that position. A degree of homology between sequences is a function of the number of matching or homologous positions shared by the sequences. An “unrelated” or “non-homologous” sequence shares less than 40% identity, though preferably less than 25% identity, with one of the sequences of the present disclosure.

A polynucleotide or polynucleotide having a certain percentage (e.g., 60%, 65%, 70%, 75%, 80%, 85%, 90%, 95%, 98%, or 99%) of “sequence identity” to another sequence means that, when aligned, that percentage of bases are the same in comparing the two sequences. This alignment and the percent homology or sequence identity can be determined using software programs known in the art, for example, those described in Ausubel et al. (2007) Curr Prot Mol Biol. Preferably, default parameters are used for alignment. One alignment program is Basic Local Alignment Search Tool (“BLAST”). Biologically equivalent polynucleotides are those having the specified percent homology and encoding a product having the same or similar biological activity.

The term “isolated” as used herein with respect to cells, nucleic acids, such as DNA or RNA, refers to molecules separated from other DNAs or RNAs, respectively that are present in the natural source of the macromolecule. The term “isolated” as used herein also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state.

As used herein the term “oligonucleotide” refers to polynucleotides such as deoxyribonucleic acid (DNA), and, where appropriate, ribonucleic acid (RNA). The term should also be understood to include, as equivalents, derivatives, variants and analogs of either RNA or DNA made from nucleotide analogs, and, as applicable to the embodiment being described, single (sense or antisense) and double stranded polynucleotides. In some embodiments, the oligonucleotide is a single stranded polynucleotide. A “nucleotide,” may refer to any molecule or portion thereof that serves as a monomer unit for forming nucleic acid molecules such as DNA or RNA (e.g., deoxyribonucleotides, ribonucleotides, cyclic nucleotides). Nucleotides contain a purine or pyrimidine base. Non-limiting examples of nucleotides include molecules that include a primary nucleobase (adenine, cytosine, guanine, thymine, and uracil), a non-primary or modified nucleobase (e.g., hypoxanthine, xanthine, 7-methylguanine, 5,6-dihydrouracil, 5-methylcytosine, 5-hydroxymethylcytodine), a purine or pyrimidine analogue, an artificial nucleobase, a nucleic acid analogue, or any derivatives thereof. For purposes of clarity, when referring herein to a nucleotide, the name of the base from which the nucleotide is derived (e.g., adenine, cytosine, guanine, thymine, uracil, etc.), is used. The terms “polynucleotide” and “oligonucleotide” are used interchangeably and refer to a polymeric form of nucleotides of any length.

The terms “pharmaceutically acceptable carrier,” “pharmaceutically acceptable diluent,” “pharmaceutically acceptable excipient,” or “pharmaceutically acceptable vehicle,” used interchangeably herein, refer to a non-toxic solid, semisolid or liquid filler, diluent, encapsulating material or formulation auxiliary of any conventional type. A pharmaceutically acceptable carrier is essentially non-toxic to recipients at the employed dosages and concentrations and is compatible with other ingredients of the formulation. The number and the nature of the pharmaceutically acceptable carriers depend on the desired administration form. The pharmaceutically acceptable carriers are known and may be prepared by methods well known in the art. See Fauli i Trillo C, “Tratado de Farmacia Galenica” (Ed. Luzan 5, S. A., Madrid, E S, 1993) and Gennaro A, Ed., “Remington: The Science and Practice of Pharmacy” 20th ed. (Lippincott Williams & Wilkins, Philadelphia, PA, US, 2003), which are incorporated by reference as if fully set forth herein. The term “prevention,” as used herein, means the administration of an immunogenic composition of the invention or of a medicament containing it in an initial or early stage of the infection, to avoid or lessen the appearance of clinical signs.

The term “DNA virus” as used herein refers to a class of viruses of vertebrate animals in which the genetic material is single stranded DNA (ssDNA) or double stranded DNA (dsDNA), and replicates using a DNA-dependent DNA polymerase. Non-limiting examples of DNA viruses include adenovirus, papillomavirus, parvovirus, herpes simplex virus, varicella-zoster virus, cytomegalovirus, Epstein-Barr virus, smallpox virus, vaccinia virus, and hepatitis B virus.

The term “RNA virus” as use herein refers to a class of viruses of vertebrate animals in which the genetic material is single stranded RNA (ssRNA) or double stranded RNA (dsRNA), and use their own RNA replicase enzymes to create copies of their genomes. Non-limiting examples of RNA viruses include rotavirus, norovirus, enterovirus, hepatovirus, rubella virus, influenzaviruses (A, B, and C), measles virus, mumps virus, hepatitis C virus, yellow fever virus, hantavirus, Zika virus, California encephalitis virus, rabies virus, ebola virus, and HIV.

The term “retrovirus” as used herein refers to a class of viruses of vertebrate animals in which the genetic material is RNA, instead of DNA. Such viruses are accompanied by a polymerase enzyme known as “reverse transcriptase,” which catalyzes transcription of viral RNA into DNA that is integrated into a host cell's genome. The resultant DNA may remain in a dormant state in an infected cell for an indeterminate period of time, or become incorporated into the cell genome and actively cause the formation of new virions. Non-limiting examples of retroviruses include HIV.

A “subject,” “individual” or “patient” is used interchangeably herein and refers to a vertebrate, for example a primate, a mammal or preferably a human. Mammals include, but are not limited to equines, canines, bovines, ovines, murines, rats, simians, humans, farm animals, sport animals and pets.

The term “treat” or “treatment” as used herein refers to the administration of an agent of the invention or of a medicament containing it to control the progression of the disease before or after clinical signs have appeared. Control of the disease progression is understood to mean the beneficial or desired clinical results that include, but are not limited to, reduction of the symptoms, reduction of the duration of the disease, stabilization of pathological states (specifically to avoid additional deterioration), delaying the progression of the disease, improving the pathological state and remission (both partial and total). The control of progression of the disease also involves an extension of survival, compared with the expected survival if treatment was not applied. Within the context of the present disclosure, the terms “treat” and “treatment” refer specifically to preventing or slowing the infection and destruction of healthy CD4+ T cells in a HIV-1 infected subject. It also refers to the prevention and slowing the onset of symptoms of the acquired immunodeficiency disease such as extreme low CD4⁺ T cell count and repeated infections by opportunistic pathogens such as Mycobacteria spp., Pneumocystis carinii, and Pneumocystis cryptococcus. Beneficial or desired clinical results include, but are not limited to, an increase in absolute naive CD4⁺ T cell count (range 10-3520), an increase in the percentage of CD4⁺ T cell over total circulating immune cells (range 1-50%), and/or an increase in CD4⁺ T cell count as a percentage of normal CD4⁺ T cell count in an uninfected subject (range 1-161%). “Treatment” can also mean prolonging survival of the infected subject as compared to expected survival if the subject did not receive any HIV targeted treatment.

The term “viral load” as used herein, refers to the amount of viral particles or fragments thereof in a biological fluid, such as blood or plasma. “Viral load” encompasses all viral particles, infectious, replicative and non-infective, and fragments thereof. Therefore, the viral load represents the total number of viral particles and/or fragments thereof circulating in the biological fluid. Viral load can be a measure of any of a variety of indicators of the presence of a virus, such as viral copy number per unit of blood or plasma, units of viral proteins or fragments thereof per unit of blood or plasma, or HIV RNA copies per milliliter of blood or plasma. RNA copies can be measured using techniques well known in the art, for example, using quantitative RT-PCR. Viral load correlates with the likelihood of a response to other viral therapies. Therefore, reducing the viral load can improve the effectiveness of other therapies.

### Oligonucleotides

The present disclosure relates to modified synthetic oligonucleotides, for example modified antisense oligonucleotides (AONs). AONs are single stranded synthetic oligonucleotides that recognize target nucleic acid sequences (e.g., RNA or DNA sequences) via Watson-Crick base pairing and cause pre- or post-transcriptional gene silencing. It is contemplated that the mechanism of action is, at least in part, RNase H cleavage of target RNA, steric hindrance of the translation machinery or prevention of RNA-RNA or RNA-protein interactions. Vickers et al., (2014) PLoS One 9, e108625; Lima et al., (2007) Mol. Pharmacol. 71:83-91; Lima et al., (2007) Mol. Pharmacol. 71:73-82. When bound to a DNA sequence, AONs prevent transcription. According to the embodiments described herein, the modified synthetic oligonucleotides (or “modified AONs”) include a plurality of nucleotides wherein at least one nucleotide is a sugar-modified nucleotide.

In certain embodiments, the modified synthetic oligonucleotides includes at least one nucleotide that is a 2′-deoxy-2′-fluoroarabinonucleotide (2′-FANA)-modified nucleotide. In such embodiments, the modified synthetic oligonucleotides described herein are referred to as 2′-FANA-modified synthetic oligonucleotides or 2′-FANA-modified AONs.

According to the embodiments described herein, a 2′-FANA-modified synthetic oligonucleotide (or other sugar-modified synthetic oligonucleotide) is designed to target a portion of a viral genome to inhibit viral expression or otherwise prevent viral transmission and infection. Thus, in some embodiments, at least a portion of the 2′-FANA-modified synthetic oligonucleotide (or other sugar-modified synthetic oligonucleotide) is complementary to a target viral nucleic acid sequence. The target viral nucleic acid may be a viral genomic ribonucleic acid (RNA) sequence in the case of an RNA virus or retrovirus, or a viral genomic deoxyribonucleic acid (DNA) sequence in the case of a DNA virus. In some embodiments, at least a portion of the 2′-FANA-modified synthetic oligonucleotide is complementary to a target nucleic acid sequence.

The 2′-FANA-modified synthetic oligonucleotide (or other sugar-modified synthetic oligonucleotide) may be designed to bind to all or a portion of a desired target viral nucleic acid sequence involved in a target virus's expression, replication, packaging, or any other sequence involved in viral transmission. For example, a 2′-FANA-modified synthetic oligonucleotide may be designed to target a viral packaging sequence to prevent proper packaging of the target virus.

The 2′-FANA-modified synthetic oligonucleotides (or other sugar-modified synthetic oligonucleotides) described herein may be used to target any virus to prevent or treat infection of host cells. Viruses that may be targeted included, but are not limited to, retroviruses (e.g., lentiviruses), herpesviruses (e.g., varicella-zoster virus, herpesviruses, Epstein-Barr virus), ebolavirus, papillomaviruses, rubulaviruses, rubiviruses, morbilliviruses, rotaviruses, noroviruses, adenoviruses, astroviruses, influenza viruses, hepaciviruses, and flaviviruses. By targeting sequences involved in transmission of such viruses, the 2′-FANA-modified synthetic oligonucleotides described herein may be used to prevent or treat infections including, but not limited to, HIV, chickenpox, ebola, flu (influenza), herpes, human papillomavirus (HPV), infectious mononucleosis, mumps, measles, rubella, shingles, viral gastroenteritis (stomach flu), viral hepatitis (Hepatitis C), viral meningitis, viral pneumonia, and/or Zika.

In some embodiments, a synthetic oligonucleotide comprising a 2′-FANA-modified nucleotide sequence according to any embodiment described herein inhibits dimerization of retroviral genomes. In certain embodiments, the 2′-FANA-modified synthetic oligonucleotides (or other sugar-modified synthetic oligonucleotides) described herein inhibits dimerization by targeting the dimerization initiation site (DIS) of a retrovirus genomic RNA molecule or a portion thereof.

Non-limiting examples of retrovirus genomic RNA molecules include an alpharetrovirus genome (e.g., avian leukemia virus), a betaretrovirus genome (e.g., mouse mammary tumor virus), a gammaretrovirus genome (e.g., murine leukemia virus, feline leukemia virus, xenotropic murine leukemia-related virus), a deltaretrovirus genome (e.g., human T-cell leukemia virus), an epsilonretrovirus genome (e.g., wall-eyed sarcoma virus), a lentivirus genome (e.g., HIV (i.e., HIV-1 or HIV-2, SIV, FIV), a spumavirus genome (e.g., human foamy virus). In one embodiment, the retrovirus genomic RNA molecule is a human immunodeficiency virus (HIV) genome.

In some embodiments, the retrovirus genomic RNA molecule is a human immunodeficiency virus (HIV) genome. HIV-1 and HIV-2 are different types of HIV. In one embodiment, the AON targets a region of HIV-1, for example, the dimerization initiation sequence (DIS). The HIV-1 DIS stem-loop consists of an approximately 35 base sequence that is located between a primer binding site and the major splice donor site, which folds into a hairpin structure with an exposed palindromic sequence flanked by 5′ and 3′ purines within its loop. This highly conserved palindrome sequence, which consists of a 5′-GCGCGC-3′, 5′-GTGCAC-3′ or 5′-GTGCGC-3′ within the DIS stem-loop is important for the formation of viral RNA dimers in vitro. According to the proposed model for dimer formation, contact between two DIS hairpins is initiated by base pairing of the self-complementary palindrome sequences to form what is known as the kissing-loop complex. Thus, according to some embodiments, a 2′-FANA-modified synthetic oligonucleotide may be designed to target the DIS to prevent dimerization and subsequent RNA packaging. The DIS of HIV is shown below:

| | HIV Dimerization Initiation Site |
|---|---|
| | Accession: 2GM0_A |
| SEQ ID NO: 1 | |
| | GACGGCTTGC TGAAGCGCGC ACGGCAAGAG GCGTC |

In some embodiments, the modified synthetic oligonucleotide comprises at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25, successive nucleotides of SEQ ID NO: 1 or a sequence complimentary thereto. In one embodiment, the modified synthetic oligonucleotide comprises at least 9 successive nucleotides of SEQ ID NO: 1 or a sequence complimentary thereto. In some embodiments, the plurality of nucleotides comprises any one of the nucleotide sequence of SEQ ID NO: 2-49 (Table 1), or an equivalent of each thereof. For purposes of the present disclosure, a molecule having a thymine or uracil at the same position is considered equivalent of any of the following sequences.

TABLE 1
Antisense oligonucleotides

| SEQ ID NO | SEQUENCE |
|---|---|
| 2. | UGUGCACUU |
| 3. | UGTGCACUU |
| 4. | UGUGCACTU |
| 5. | GCCGUGTGCACTTCAGCA |
| 6. | GCCGTGUGCACUTCAGCA |
| 7. | UUGCCGUGUGCACUUCAGAA |
| 8. | UUGCCGTGTGCACTUCAGCA |
| 9. | UUGCCGTGUGCACTTCAGCA |
| 10. | UGCCGUGTGCACUUCAGCAA |
| 11. | UGCCGTGTGCACTTCAGCAA |
| 12. | UGCCGUGTGCACUUCAGCA |
| 13. | UUGCCGUGTGCACUUCAGCAA |
| 14. | UTGCCGUGUGCACTUCAGCAA |
| 15. | UUGCCGTGUGCACUTCAGCAA |
| 16. | CUCGCCTCTTGCCG |
| 17. | CUCGCCUCUTGCCG |
| 18. | CUCGCCUCTTGCCG |
| 19. | CUCGCCUCUUGCCG |
| 20. | CTCGCCUCUTGCCG |
| 21. | CAGCAAGCCGAG |
| 22. | CGCCTCUUGCCGUGUGCACUU |
| 23. | GCCUCUTGCCGTGTGCACUU |
| 24. | CGCCTCTUGCCGTGTGCACUU |
| 25. | CGCCUCUTGCCGUGTGCACUU |
| 26. | CGCCUCUTGCCGUGUGCACTU |
| 27. | CUCTTGCCGUGUGCACUUC |
| 28. | CUCTTGCCGUGTGCACUU |
| 29. | CTCUTGCCGUGUGCACTU |
| 30. | CUCTUGCCGUGTGCACUU |
| 31. | UGUGCACUUCAGCAAGCCGA |
| 32. | GUGUGCACTTCAGCAAGCC |
| 33. | GUGUGCACUTCAGCAAGCC |
| 34. | UGUGCACTUCAGCAAGCC |
| 35. | UGAGCTCUUCGTCGCTGTCUC |
| 36. | UGAGCTCTTCGTCGCUGUCU |
| 37. | GUCTGAGGGATCUCUAGTUAC |
| 38. | UCUGAGGGATCTCTAGUUAC |
| 39. | GUGAGCTCUUCGTCGCTGTCUC |
| 40. | UGAGCTCUUCGTCGC |
| 41. | CUGAGGGTCTCTAGUU |
| 42. | GUCUGAGGGATCTCTAGUUAC |
| 43. | TGTGCACTT |
| 44. | CTTGCCGTGTGCACTTCAGCAAGCCG |
| 45. | CTCGCCTCTTGCCG |
| 46. | CTCGCCTCTTGCCGTGTGCACTT |
| 47. | TGTGCACTTCAGCAAGCCGAG |
| 48. | UGCCGUGUGCACUUCAGCAA |
| 49. | TGCCGTGTGCACTTCAGCAA |

In some embodiments, the modified synthetic oligonucleotide has at least 60%, at least 65%, at least 70%, at least 75%, at least 80%, at least 85%, at least 90%, at least 95%, at least 98%, or at least 99%) sequence identity to any one of SEQ ID Nos. 1-49.

The modified synthetic oligonucleotide sequences of the present disclosure include one or more nucleotides having a modified sugar moiety. Non-limiting examples of modified sugar moieties include 2′-O-methyl (2′-OMe) nucleotide, 2′-fluoro (2′-F) nucleotide, 2′-O-methoxyethyl (2′-MOE) nucleotide, an arabinonucleotide (ANA), 2′-deoxy-2′-fluoroarabinonucleotide (2′-FANA), 2'S-F-ANA, a 4′thio nucleotide, and a bicyclic sugar moiety. In one embodiment, the modified sugar moiety is a 2′FANA nucleotide.

The modified synthetic oligonucleotide sequence may include a modified sugar moiety for all or a portion of the nucleotides in the sequence. In some embodiments, the synthetic oligonucleotides comprise at least one unmodified nucleotide, for example, between 2 and 10 unmodified nucleotides. In some embodiments, the modified synthetic oligonucleotides comprise between 1 and 20 nucleotides. In some embodiments, the synthetic oligonucleotides comprise 1, 2, 3, 4, 5, 6, 7, 8, 9, 10, 11, 12, 13, 14, 15, 16, 17, 18, 19, 20, or more than 20 unmodified nucleotides. In certain embodiments, the synthetic oligonucleotide does not have any unmodified nucleotides, having only modified sugar moiety nucleotides.

In some embodiments, the at least one unmodified nucleotide is located within the modified synthetic oligonucleotide between nucleotides comprising modified sugar moieties (“sugar-modified nucleotides” or “2′-FANA-modified nucleotides”). For example, a modified synthetic oligonucleotide may comprise a string of at least 2, at least 3, at least 4, at least 5, at least 6, at least 7, at least 8, at least 9, at least 10, or more sugar-modified or 2′-FANA-modified nucleotides, followed by at least 1, at least 2, at least 3, at least 4, at least 5, at least 6, at least 7, at least 8, at least 9, at least 10, or more unmodified nucleotides, followed by another string of at least 4, at least 5, at least 6, at least 7, at least 8, at least 9, at least 10 or more sugar-modified or 2′-FANA-modified nucleotides, in any combination thereof. In certain embodiments, when one or more unmodified nucleotides are flanked by the sugar-modified or 2′-FANA-modified nucleotides, the unmodified nucleotide(s) may be referred to as a “nucleotide gap sequence.” The nucleotide gap sequence may consist of 1, 2, 3, 4, 5, 6, 7, 8, 9, 10, 11, 12, 13, 14, 15, 16, 17, 18, 19, 20, or more than 20 unmodified nucleotides. A modified synthetic oligonucleotide in accordance with the embodiments described herein may include a single nucleotide gap sequence, or may include more than one nucleotide gap sequence within the same molecule. Further, when the sugar-modified or 2′-FANA-modified nucleotides flank a nucleotide(s) they can be of the same length or different lengths. For example, the modified synthetic oligonucleotide may comprise 8 nucleotides comprising a first string of modified sugar moieties, followed by 6 nucleotides, followed by a second string of nucleotides comprising modified sugar moieties, but wherein the number of modified nucleotides in the second string differs from the number of modified sugar moieties in the first string.

In certain embodiments, the modified synthetic oligonucleotide comprises a sugar modified nucleotide sequence, for example, 2′-FANA-modified nucleotide sequence, flanking a series of unmodified nucleotide residues of variable length, wherein the ribonucleotide gap sequence comprises between 2 and 10 unmodified nucleotides. In some embodiments, the modified synthetic oligonucleotide comprises (i) a first sugar modified nucleotide sequence (for example, 2′-FANA-modified nucleotide sequence) comprising between 1 and 10 sugar modified nucleotides, (ii) an unmodified nucleotide sequence comprising between 1 and 10 nucleotides, followed by (ii) a second sugar modified nucleotide sequence comprising between 1 and 10 sugar modified nucleotides, and optionally repeating the alternating pattern of a sugar modified nucleotide sequence, a nucleotide sequence, and a modified nucleotide sequence between 1 and 10 times.

Non-limiting examples of modified synthetic oligonucleotides according to the embodiments described herein include, but are not limited to, the formulas shown in Table 2 below:

TABLE 2

| No. of | |
|---|---|
| nucleotides | Formula |
| 9 | XXXXXXXXX |
| | XXXXXXXXX |
| | XXXXXXXXX |
| | XXXXXXXXX |
| | XXXXXXXXX |
| | XXXXXXXXX |
| | XXXXXXXXX |
| | XXXXXXXXX |
| | XXXXXXXXX |
| | XXXXXXXXX |
| | XXXXXXXXX |
| | XXXXXXXXX |
| | XXXXXXXXX |
| | XXXXXXXXX |
| | XXXXXXXXX |
| 12 | XXXXXXXXXXXX |
| | XXXXXXXXXXXX |
| | XXXXXXXXXXXX |
| | XXXXXXXXXXXX |
| | XXXXXXXXXXXX |
| | XXXXXXXXXXXX |
| | XXXXXXXXXXXX |
| | XXXXXXXXXXXX |
| | XXXXXXXXXXXX |
| | XXXXXXXXXXXX |
| | XXXXXXXXXXXX |
| | XXXXXXXXXXXX |
| 14 | XXXXXXXXXXXXXX |
| | XXXXXXXXXXXXXX |
| | XXXXXXXXXXXXXX |
| | XXXXXXXXXXXXXX |
| | XXXXXXXXXXXXXX |
| 15 | XXXXXXXXXXXXXXX |
| 17 | XXXXXXXXXXXXXXXXX |
| 18 | XXXXXXXXXXXXXXXXXX |
| | XXXXXXXXXXXXXXXXXX |
| | XXXXXXXXXXXXXXXXXX |
| | XXXXXXXXXXXXXXXXXX |
| | XXXXXXXXXXXXXXXXXX |
| | XXXXXXXXXXXXXXXXXX |
| | XXXXXXXXXXXXXXXXXX |
| | XXXXXXXXXXXXXXXXXX |
| 19 | XXXXXXXXXXXXXXXXXXX |
| | XXXXXXXXXXXXXXXXXXX |
| 20 | XXXXXXXXXXXXXXXXXXXX |
| | XXXXXXXXXXXXXXXXXXXX |
| | XXXXXXXXXXXXXXXXXXXX |
| | XXXXXXXXXXXXXXXXXXXX |
| 21 | XXXXXXXXXXXXXXXXXXXXX |
| | XXXXXXXXXXXXXXXXXXXXX |
| | XXXXXXXXXXXXXXXXXXXXX |
| | XXXXXXXXXXXXXXXXXXXXX |
| | XXXXXXXXXXXXXXXXXXXXX |
| | XXXXXXXXXXXXXXXXXXXXX |
| | XXXXXXXXXXXXXXXXXXXXX |
| | XXXXXXXXXXXXXXXXXXXXX |
| | XXXXXXXXXXXXXXXXXXXXX |
| | XXXXXXXXXXXXXXXXXXXXX |

The formulas shown in Table 2 may be applied to any of SEQ ID NOs. 2-49, or a portion thereof, wherein X represents a nucleotide (A, C, G, T, or U), and wherein bold and underlined nucleotides represent sugar-modified or 2′-FANA-modified nucleotides. Exemplary sequences of modified synthetic oligonucleotides in accordance with the present disclosure are shown in FIG. 8.

The modified synthetic oligonucleotides of the present disclosure may further comprise internucleotide linkages between the plurality of nucleotides comprising phosphodiester bonds, phosphotriester bonds, phosphorothioate bonds (5′O—P(S)O-3O—, 5′S—P(O)O-3′-O—, and 5′O—P(O)O-3′S—), phosphorodithioate bonds, Rp-phosphorothioate bonds, Sp-phosphorothioate bonds, boranophosphate bonds, methylene bonds(methylimino), amide bonds (3′-CH₂—CO—NH-5′ and 3′-CH₂—NH—CO-5′), methylphosphonate bonds, 3′-thioformacetal bonds, (3′S—CH₂—O5′), amide bonds (3′CH₂—C(O)NH-5′), phosphoramidate groups, or any combination thereof.

In some embodiments, the overall length of the modified synthetic oligonucleotide of the disclosure is about 40 or fewer nucleotide residues, about 30 or fewer nucleotide residues, about 25 or fewer nucleotide residues, about 20 or fewer nucleotide residues, about 15 or fewer nucleotide residues, or about 10 or fewer nucleotide residues. In further embodiments, the overall length is about 5 to about 40, about 10 to about 35, about 15 to about 30, or about 20 to about 25 nucleotide residues. In some embodiments, the modified synthetic oligonucleotides comprise between about 8 and about 25 nucleotides. In some embodiments, the modified synthetic oligonucleotides comprise between 15 and 21 nucleotides. In still further embodiments, the overall length is 5 nucleotide residues, 6 nucleotide residues, 7 nucleotide residues, 8 nucleotide residues, 9 nucleotide residues, 10 nucleotide residues, 11 nucleotide residues, 12 nucleotide residues, 13 nucleotide residues, 14 nucleotide residues, 15 nucleotide residues, 16 nucleotide residues, 17 nucleotide residues, 18 nucleotide residues, 19 nucleotide residues, 20 nucleotide residues, 21 nucleotide residues, 22 nucleotide residues, 23 nucleotide residues, 24 nucleotide residues, 25 nucleotide residues, 26 nucleotide residues, 27 nucleotide residues, 28 nucleotide residues, 29 nucleotide residues or 30 nucleotide residues. In some embodiments, the overall length is 9 nucleotide residues. In some embodiments, the overall length is 12 nucleotide residues. In some embodiments, the overall length is 14 nucleotide residues. In other embodiments, the overall length is 15 nucleotide residues. In other embodiments, the overall length is 18 nucleotide residues. In some embodiments, the overall length is 20 nucleotide residues. In other embodiments, the overall length is 21 nucleotide residues.

In some embodiments, the present disclosure provides a composition comprising at least one of the synthetic oligonucleotides described herein. In some embodiments, the composition further comprises a pharmaceutically acceptable excipient, diluent, carrier, or any combination thereof.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The composition may comprise a pharmaceutically acceptable excipient, a pharmaceutically acceptable salt, diluents, carriers, vehicles and such other inactive agents well known to the skilled artisan. Vehicles and excipients commonly employed in pharmaceutical preparations include, for example, talc, gum Arabic, lactose, starch, magnesium stearate, cocoa butter, aqueous or non-aqueous solvents, oils, paraffin derivatives, glycols, etc. Solutions can be prepared using water or physiologically compatible organic solvents such as ethanol, 1,2-propylene glycol, polyglycols, dimethylsulfoxide, fatty alcohols, triglycerides, partial esters of glycerine and the like. Compositions may be prepared using conventional techniques that may include sterile isotonic saline, water, 1,3-butanediol, ethanol, 1,2-propylene glycol, polyglycols mixed with water, Ringer's solution, etc. In one aspect, a coloring agent is added to facilitate in locating and properly placing the composition to the intended treatment site.

Compositions may include a preservative and/or a stabilizer. Non-limiting examples of preservatives include methyl-, ethyl-, propyl-parabens, sodium benzoate, benzoic acid, sorbic acid, potassium sorbate, propionic acid, benzalkonium chloride, benzyl alcohol, thimerosal, phenylmercurate salts, chlorhexidine, phenol, 3-cresol, quaternary ammonium compounds (QACs), chlorbutanol, 2-ethoxyethanol, and imidurea.

To control tonicity, the composition can comprise a physiological salt, such as a sodium salt. Sodium chloride (NaCl) is preferred, which may be present at between 1 and 20 mg/ml. Other salts that may be present include potassium chloride, potassium dihydrogen phosphate, disodium phosphate dehydrate, magnesium chloride and calcium chloride.

Compositions may include one or more buffers. Typical buffers include: a phosphate buffer; a Tris buffer; a borate buffer; a succinate buffer; a histidine buffer; or a citrate buffer. Buffers will typically be included at a concentration in the 5-20 mM range. The pH of a composition will generally be between 5 and 8, and more typically between 6 and 8 e.g. between 6.5 and 7.5, or between 7.0 and 7.8.

The composition can be administered by any appropriate route, which will be apparent to the skilled person depending on the disease or condition to be treated. Typical routes of administration include intravenous, intra-arterial, intramuscular, subcutaneous, intracranial, intranasal or intraperitoneal.

In some embodiments, the composition may include a cryoprotectant agent. Non-limiting examples of cryoprotectant agents include a glycol (e.g., ethylene glycol, propylene glycol, and glycerol), dimethyl sulfoxide (DMSO), formamide, sucrose, trehalose, dextrose, and any combinations thereof.

The composition can be included in an implantable device. Suitable implantable devices contemplated by this invention include intravascular stents (e.g., self-expandable stents, balloon-expandable stents, and stent-grafts), scaffolds, grafts, and the like. Such implantable devices can be coated on at least one surface, or impregnated, with a composition capable of treating or preventing a retroviral infection, for example HIV.

One aspect of the present disclosure provides methods of inhibiting expression of a retrovirus comprising delivering a synthetic oligonucleotide described herein to a cell infected with a retrovirus. The synthetic oligonucleotide can be delivered by any suitable method to allow for uptake of the synthetic oligonucleotides by the cell without the use of any transfection reagent and/or additives. In certain aspects, the delivery method includes a technique-based transfection method including, but not limited to, electroporation or microinjection. In other aspects, the delivery method is a gymnotic delivery method.

In one embodiment, the cell is part of a population of cultured cells (i.e., in vitro). In another embodiment, the cell is part of a population of cells of a subject (i.e., in vivo). For example, the synthetic oligonucleotide may be delivered to an in vivo cell or an in vivo population of cells that form a tissue or organ in a subject for the purpose of inhibiting retroviral expression or to treat or prevent retroviral infection. Alternatively, the synthetic oligonucleotide may be delivered to a cultured cell or a population of cultured cells for the purpose of conducting experiments to study its effect on a particular type of cell.

One aspect of the present disclosure provides methods for treating or preventing a viral infection in a patient in need thereof, comprising administering to the patient an effective amount of a composition comprising any of the synthetic oligonucleotides described herein. Non-limiting examples of a viral infection include a retroviral infection, chickenpox, ebola, flu (influenza), herpes, human papillomavirus (HPV), infectious mononucleosis, mumps, measles, rubella, shingles, viral gastroenteritis (stomach flu), viral hepatitis (Hepatitis C), viral meningitis, viral pneumonia, and/or Zika.

Another aspect of the present disclosure provides methods for treating or preventing a retroviral infection in a patient in need thereof, comprising administering to the patient an effective amount of a composition comprising any of the synthetic oligonucleotides described herein.

The compositions can be administered to a patient by any suitable mode and route. Non-limiting examples include internal, pulmonary, rectal, nasal, vaginal, lingual, intravenous, intraarterial, intramuscular, intraperitoneal, intracutaneous and subcutaneous routes. Compositions may also be suitable for transdermal delivery as part of a cream, gel, or patch. Other dosage forms include tablets, capsules, pills, powders, aerosols, suppositories, parenterals, and oral liquids, including suspensions, solutions and emulsions. Sustained release dosage forms may also be used.

As used herein, the term “retroviral infection” is inclusive of any viral infection that utilizes reverse transcriptase in the viral replication cycle and therefore is susceptible to the antiviral activity induced by the synthetic oligonucleotides. The term “retrovirus” is specifically inclusive of human immunodeficiency virus (HIV-1 and HIV-2) and simian immunodeficiency virus (SIV). Additional non-limiting examples of retroviruses include bovine immunodeficiency virus (BIV), caprine encephalitis-arthritis virus (CAEV), equine infectious anemia virus (EIAV), feline immunodeficiency virus (FIV), goat leukoencephalitis virus (GLV), Jembrana virus (JDV), maedi/visna virus (MVV), and progressive pneumonia virus (PPV). The HIV can be type 1 (HIV-1) or type 2 (HIV-2). The HIV can be from any HIV Glade (e.g., A-G), strain or variant, including, for example, HIV-1:ARV-2/SF-2, HIV-1:BRU (LAI), HIV-1:CAM1, HIV-1:ELI, HIV-1:HXB2, HIV-1:IIIB, HIV-1:MAL, HIV-1:MN, HIV-1:NDK, HIV-1:PV22, HIV-1:RF, HIV-1:U455, and HIV-1:Z2. Also encompassed are viruses such as hepatitis B virus (HBV) that although not technically classified as retroviruses, nonetheless utilize a reverse transcriptase. In one embodiment, the retroviral infection is caused by HIV, for example, HIV-1, HIV-2, or combination thereof.

In some embodiments, the synthetic oligonucleotides are administered to the subject for a period effective to reduce viral load by at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 20%, at least about 25%, at least about 30% at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 97%, at least about 98%, at least about 99%, or by 100%.

In some embodiments, the treating or preventing of a viral infection is induced by RNase H activity, steric hindrance, or a combination thereof. It is contemplated when the synthetic oligonucleotide comprises only modified sugar moiety nucleotides RNase H does not recognize the hybridized viral sequence and the synthetic oligonucleotide inhibits viral infection through steric blocking.

In some embodiments, the synthetic oligonucleotides are administered to the subject with at least one additional anti-viral drug, for example, an anti-retroviral drug. Non-limiting examples of anti-retroviral drugs include, lamivudine, zidovudine, stavudine, nevirapine, abacavir, didanosine, ganciclovir, zalcitabine, efavirenz, delaviridine, nelfinavir, ritonavir, indinavir, saquinavir, amprenavir, lopinavir, and any combination thereof.

In some embodiments, the synthetic oligonucleotides and the anti-retroviral drug are administered simultaneously. In other embodiments, the synthetic oligonucleotides and the anti-retroviral drug are administered sequentially.

In some embodiments, the compositions comprising synthetic oligonucleotides effectuates a reduced viral load for a period of about 1 week, 2 weeks, 3 weeks, 4 weeks, 5 weeks, 6 weeks, 7 weeks, 8 weeks, 9 weeks, 10 weeks or more after administration.

In some embodiments, the compositions comprising synthetic oligonucleotides of the present disclosure inhibit dimerization initiation of viral RNAs, increase viral RNA cleavage, or both. The viral RNA cleavage can be mediated, at least in part to RNase H1 activity.

In some embodiments, the compositions comprising synthetic oligonucleotides of the present disclosure are gymnotically administered to the patient.

In other embodiments, the compositions comprising synthetic oligonucleotides are administered to the patient along with a transfection reagent or other transfection method. Non-limiting examples of transfection reagents and methods include gene gun, electroporation, nanoparticle delivery (e.g., poly(alkylcyanoacrylate) nanoparticles, PEG-coated nanoparticles, polyisohexylcyanoacrylate (piHCA) nanoparticles) cationic lipids and/or polymers. Specific examples include in vivo-jetPEI® (Polyplus, New York, NY, USA), X-tremeGENE reagents (Roche Life Sciences, Indianapolis, IN, USA), 1,2-dioleoyl-sn-glycero-3-phosphatidylcholine (DOPC) neutral liposome, cyclodextrin-containing polymer CAL101, and lipid nanoparticles.

### EXAMPLES

The following examples are intended to further illustrate certain embodiments of the disclosure. The examples are put forth so as to provide one of ordinary skill in the art and are not intended to limit its scope.

### Example 1: Generation of HIV-1 Targeting Antisense Oligonucleotides

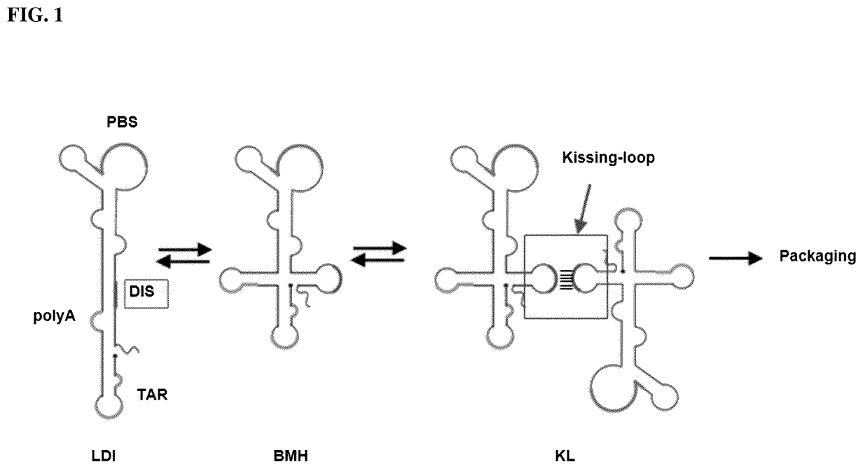

Human immunodeficiency virus-1 (HIV-1) viral particles contain two copies of genomic RNA, which form dimers via intermolecular interactions. The dimerization process is initiated by the formation of a kissing-loop dimer through base pairing of the palindromic loop sequence within dimerization initiation site (DIS). As shown in FIG. 1, the 5′-UTR of the HIV-1 RNA genome contains replication signals that are required in various steps in the replication cycle, including dimerization initiation site (DIS). Dimerization is initiated by conformation change of 5′-UTR from LDI (long-distance interaction) to BMH (branched multiple hairpin). This allows the DIS loop to intermolecular base pairs between two RNA genomes, forming a kissing-loop (KL) dimer and subsequent RNA packaging.

Antisense oligonucleotides (AONs) are single-stranded synthetic oligonucleotides that recognize target RNAs via Watson-Crick base pairing and cause post-translational inhibition. The mechanisms are believed to be RNase H cleavage of target RNA, steric hindrance of the translation machinery or prevention of RNA-RNA or RNA-protein interactions. While AONs offer promising solutions for variety of human diseases in preclinical studies, many of which are currently in clinical studies, a number of challenges still hamper their translation from the bench to the bedside. The most significant of these challenges include target accessibility, off target effects, poor extracellular and intracellular stability and effective delivery into target cells.

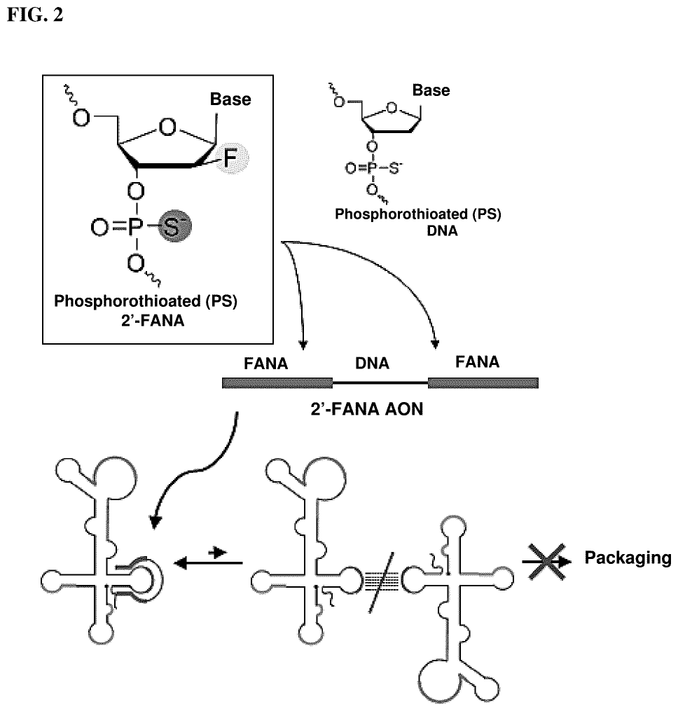

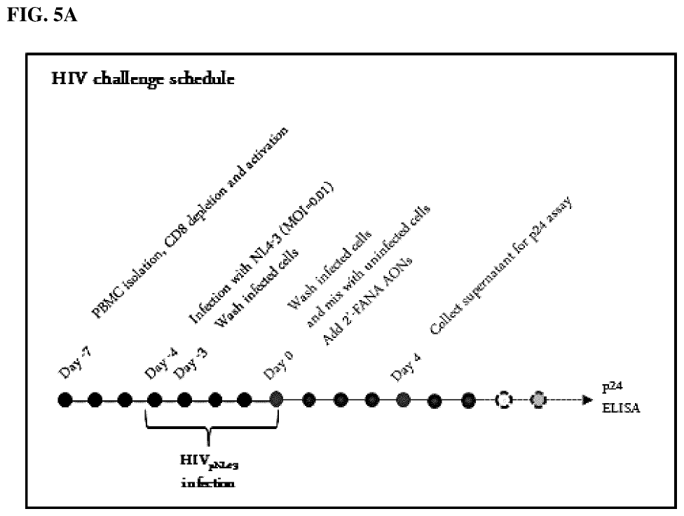

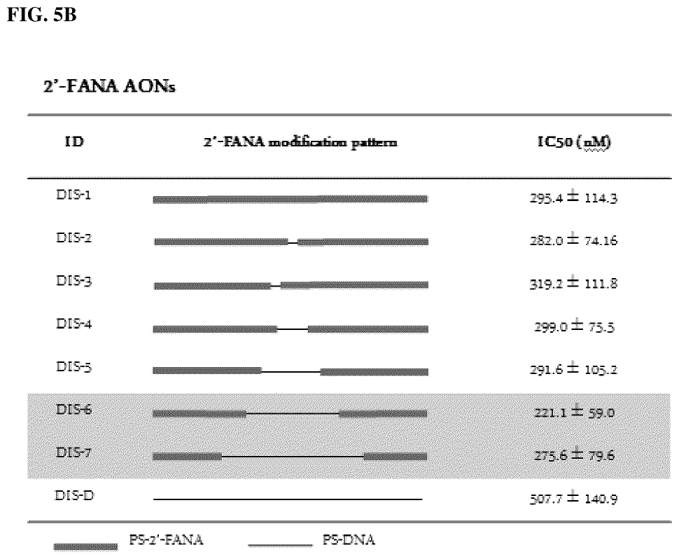

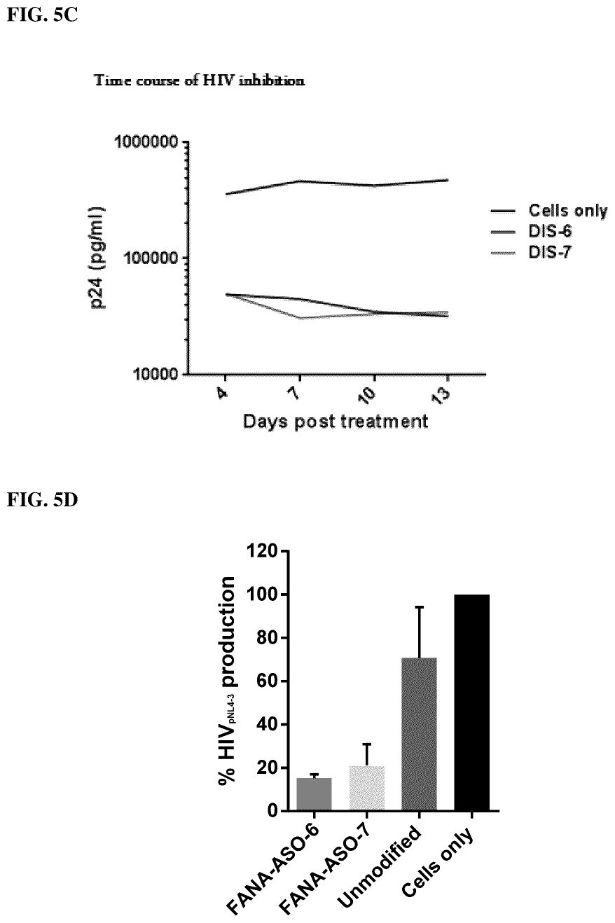

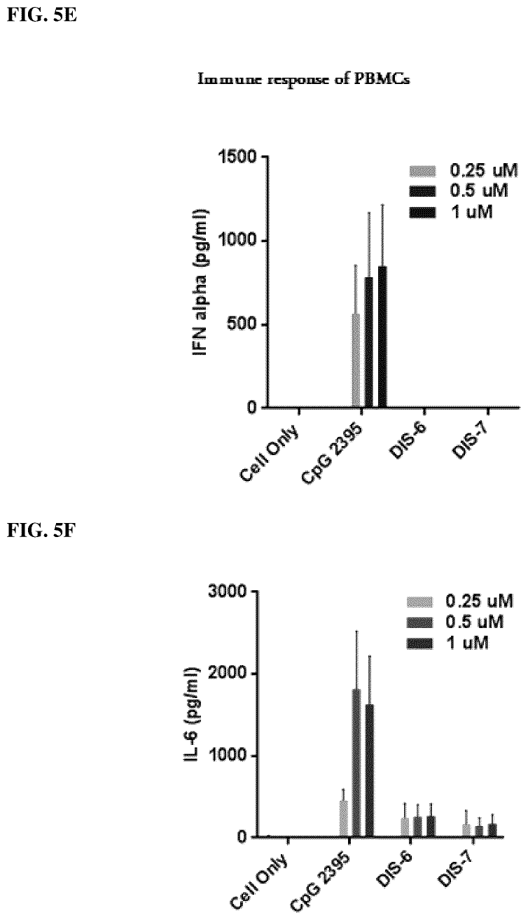

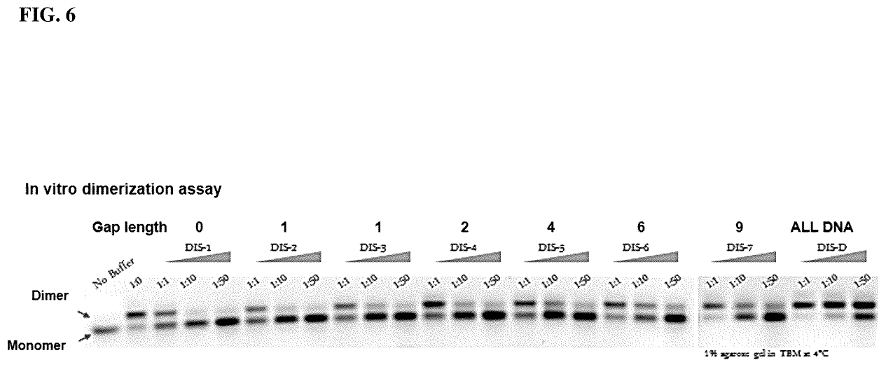

2′-dexoy-2′-fluoro-β-D-arabinonucleic acid modified antisense oligonucleotides (2′-FANA AONs) were generated as depicted by FIG. 2 were generated. Briefly, these synthetic oligonucleotides comprised F-ANA domains on either end of a DNA “gap.” The nucleotides were linked via phosphorothioated (PS) linkages. Representative 2′-FANA AONs that were generated are depicted in FIG. 5B. As shown, AONs were generated containing a range of DNA gap lengths. For example, DIS-1 is an AON comprising all 2′-FANA nucleotides with no DNA gap, while DIS-D comprises only unmodified nucleotides. DIS-2 through DIS-7 represent 2′-FANAs comprising between 1 and 9 nucleotides flanked on either end by 2′-FANA modified nucleotides. The sequences of DIS-1 through DIS-7 (and DIS-D) are shown below:

| | | | Nucleo- | |
|---|---|---|---|---|
| | Sequence and | | tide | |
| Oligo | Modification | | Gap | SEQ |
| ID | Patterns | Length | length | ID |
| DIS-1 | UGCCGUGUGCACUUCAGCAA | 20 | 0 | 48 |
| DIS-2 | UGCCGUGUGCACUUCAGCAA | 20 | 1 | 48 |
| DIS-3 | UGCCGUGUGCACUUCAGCAA | 20 | 1 | 48 |
| DIS-4 | UGCCGUGUGCACUUCAGCAA | 20 | 2 | 48 |
| DIS-5 | UGCCGUGTGCACUUCAGCAA | 20 | 4 | 10 |
| DIS-6 | UGCCGUGTGCACUUCAGCAA | 20 | 6 | 10 |
| DIS-7 | UGCCGTGTGCACTTCAGCAA | 20 | 9 | 11 |
| DIS-D | TGCCGTGTGCACTTCAGCAA | 20 | All | 49 |

As shown in FIG. 5B, all of the 2′-FANA-modified sequences (DIS-1 through DIS-7) showed enhanced binding efficiencies (IC₅₀ (nM) between 221.1±59.0 and 319.2±111.8) as compared to DIS-D (IC₅₀ (nM) of 507.7±140.9) which had no modifications. Of the 2′-FANA AONs tested, DIS-6 and DIS-7 had the highest binding efficiencies of 221.1±59.0 and 275.6±79.6, respectively. These AONs had high affinity to complementary RNA, were resistant to exo- and endonculeases, have a dual mechanism of action (steric block and/or RNase H activation), and increased target specificity.

### Example 2: Gymnotic Delivery Antisense Oligonucleotides

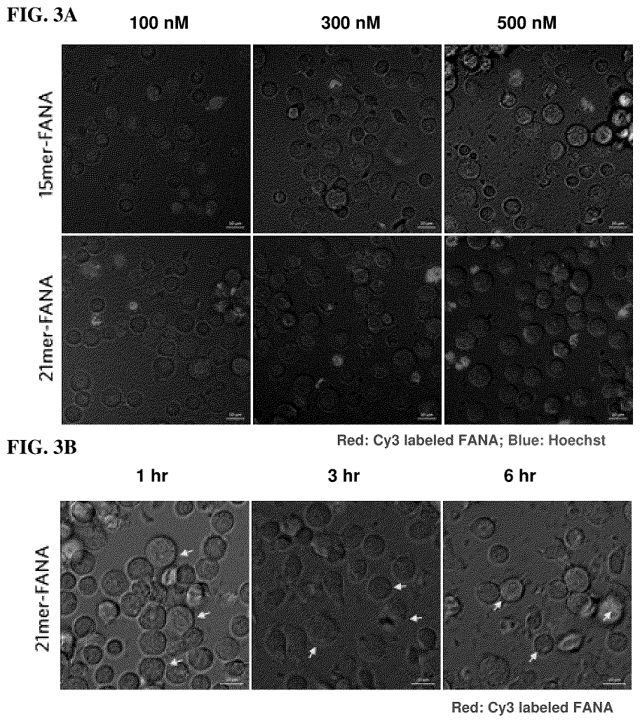

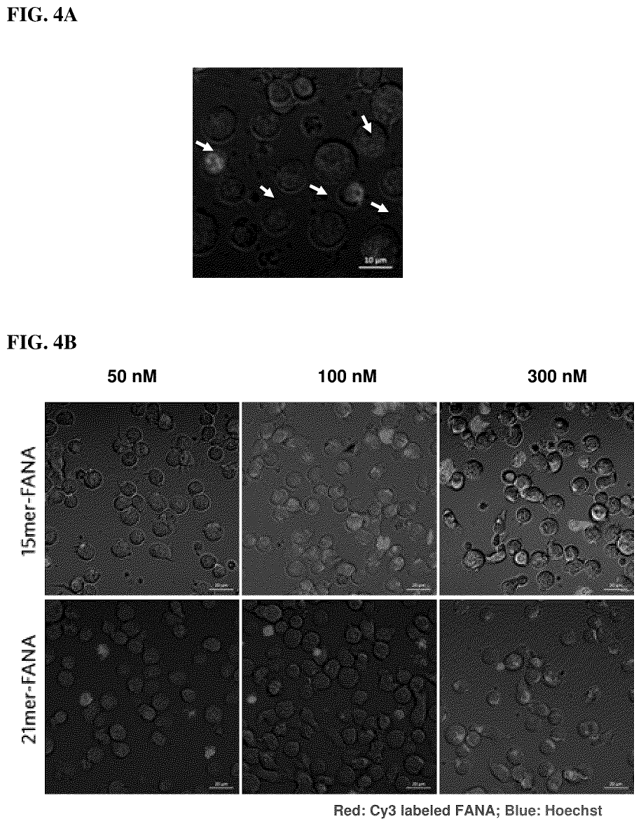

Cy3 labeled 15-mer and 21-mer 2′-FANA modified AONs generated above were gymnotically delivered to peripheral blood mononuclear cells (PBMCs) and CEM cells in various concentrations. Cells were incubated with AONs for 4 hours at 37° C. Real-time live cell images were collected using 40× magnification on confocal microscopy. Efficient cellular update of the 2′-FANA modified AONs was observed within an hour after AON treatment. As shown in FIG. 3A, cells incubated with 100 nM, 300 nM, or 500 nM 21-mer-FANA showed an increase in delivery of the modified AONs 4 hours after delivery. On the other hand, the 15mer-FANA showed minimal gymnotic delivery of the AON (FIG. 3, top left panel). FIG. 3B depicts representative images from a time course experiment, representing 1 hr, 3 hr, and 6 hour following incubation with AONs. As shown, an increase in Cy3 positive cells was observed over the time course. Arrows identify cells that have taken up the AONs in the absence of any transfection reagent. FIG. 4A is a representative higher magnification confocal image of cytoplasmic uptake of Cy3-AONs in PBMCs after 4 hours of incubation. Arrows again identifying cells that have taken up the AONs in the absence of any transfection reagent. Similarly, CEM cells showed an increase in delivery of the modified AONs for both oligonucleotides tested and at different concentrations FIG. 4B.

Together, these data show that the 2′-FANA AONs can be successfully delivered to even hard to transfect cells, such as PBMCs, even in the absence of a transfection reagent.

### Example 3: Inhibition of HIV-1 Expression by 2′-FANA AONs

Next, two oligonucleotides with different “gap” DNA regions (DIS-6 and DIS-7) were tested to determine differences in the HIV-1 inhibitory effect of the variously 2′-FANA modified AONs in HIV-1 infected PBMCs. Briefly, PBMCs were isolated from human peripheral blood from healthy donors and activated in T cell activation media. After three days activation, PBMCs were infected with HIVpN4-3 at MOI of 0.01. On the following day, infected cells were washed three times with PBS, suspend in fresh media and incubated for three days. Infected cells were washed and mixed with the same number of uninfected cells. 2′-FANA AONs were added to cells at various concentrations (0.1-1.6 μM for dose response assay and 3 M for time course assay). Cells were then incubated at 37° C. and cell supernatant was collected and stored in −20° C. until the assay. (PerkinElmer, Waltham, MA, USA). FIG. 5A. As shown in FIGS. 5C and 5D, the 2′-FANA AONs gymnotically delivered to PBMCs showed strong inhibition of HIVpNL4-3 expression. The inhibitory effect was dose dependent and lasted as long as two weeks after treatment (data not shown).

Synthetic oligonucleotides have been reported to nonspecifically active innate inflammatory cytokine production, for example, tumor necrosis factor-α (IFN-α), interleukin-6 (IL-6), and interleukin-12 (IL-12) as well as interferon (IFN)-responsive genes, and this, in turn, can trigger undesirable cellular toxicity. Therefore, immune responses of PBMCs to 2′-FANA AON treatment were also measured by detection. No significant differences in IFN-α (FIG. 5E) or IL-6 (FIG. 5F) were observed in cells incubated with DIS-6 or DIS-7 AONs as compared to control cells. On the other hand, cells incubated with the CpG 2395 oligonucleotide, which is known to induce strong immunostimulatory effects, showed significant increases in both IFN-α (FIG. 5E) or IL-6 (FIG. 5F). As such, the AONs of the present disclosure do not appear to active an undesirable inflammatory response.

In vitro dimerization assays were also performed to determine whether the 2′-FANA AONs inhibit target RNA dimerization. 2′-FANA AONs were mixed with target RNA transcript. The mixtures were incubated at 95° C. for 3 min and then snap-cooled on ice. After adding 5× dimerization buffer (final: 50 mM Na-cacodylate; pH 7.5, 250 mM KCl, 5 mM MgCl2), mixtures were incubated at 37° C. for 30 min and then run on a 1% agarose gel in Tris-borate-magnesium (TBM) buffer at 4° C. After running, gels were stained with ethidium bromide and images were captured with an Eagle Eye II system (Agilent Technologies, Santa Clara, CA, USA), As shown in FIG. 6, AONs DIS-1 through DIS-7 all inhibited dimerization formation, particularly at 1:10 and 1:50 concentrations of HIV-1 mRNA to AON.

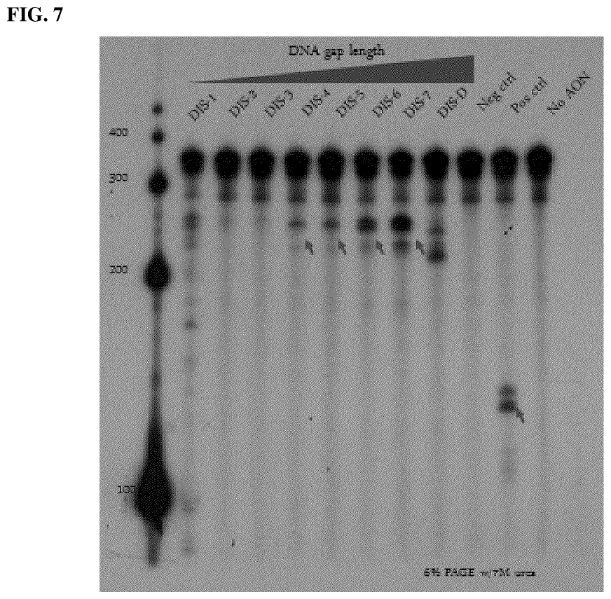

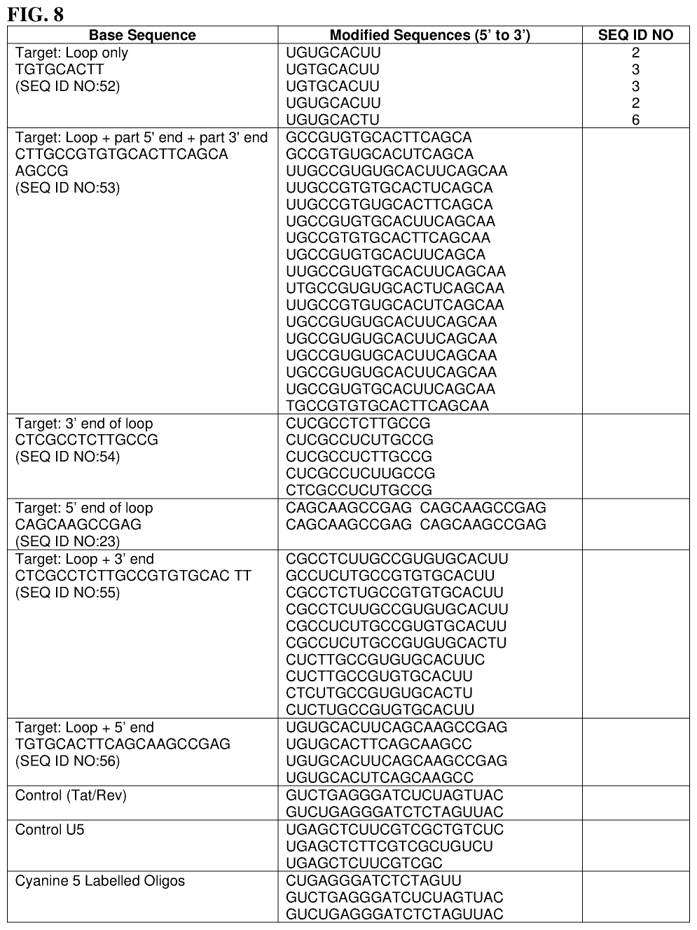

In addition, since it is known that gapmer type AONs mediate RNase H1 cleavage of target RNAs, cleavage of target RNA by the 2′-FANA AONs was also examined. 2′-FANA AONs were mixed with 5′-32P-labeled target RNA transcript in annealing buffer (1×: 10 mM Tris (pH 7.5) 50 mM NaCl, 1 mM EDTA). Samples were heated at 90° C. for 3 min and slowly cooled to room temperature. After adding human RNase H, the reaction mixture was incubated at 37° C. for 1 h. As shown in FIG. 7, DIS-4, DIS-5, DIS-6, and DIS-7 AONs showed an increase in target RNA cleavage.

Together, these data show that the 2′-FANA AONs can be gymnotically delivered into PBMCs without any transfection reagent. These studies also demonstrate that strong and long lasting inhibition of HIV can be achieved by 2′-FANA AON treatment. In addition, the inhibitory effect of 2′-FANA AONs is likely to be attributed to both RNase H1 activation and dimerization inhibition. As such, 2′-FANA AONs represent promising novel drug candidates for antiretroviral therapy.

From the foregoing, it will be appreciated that specific embodiments of the invention have been described herein for purposes of illustration, but that various modifications may be made without deviating from the scope of the invention. Accordingly, the invention is not limited except as by the appended claims.

### REFERENCES

1. Vickers, T. A. and Crooke, S. T. (20 14) Antisense oligonucleotides capable of promoting specific target mRNA reduction via competing RNase HI-dependent and independent mechanisms. PloS One, 9, e 108625.
2. Lima, W. F., Rose, J. B., Nichols, J. G., Wu, H., Migawa, M. T., Wyrzykiewicz, T. K., Siwkowski, A. M. and Crooke, S. T. (2007) Human RNase H1 discriminates between subtle variations in the structure of the heteroduplex substrate. Mol Pharmacol, 7 1, 83-91.
3. Lima, W. F., Rose, J. B., Nichols, J. G., Wu, H., Migawa, M. T., Wyrzykiewicz, T. K., Vasquez, G., Swayze, E. E. and Crooke, S. T. (2007) The positional influence of the helical geometry of the heteroduplex substrate on human RNase HI catalysis. Mol Pharmacol, 71, 73-82.
4. Chan, J. H., Lim, S. and Wong, W. S. (2006) Anti sense oligonucleotides: from design to therapeutic application. Clin Exp Pharmacol Physiol, 33, 533-540.
5. Geary, R. S., Norris, D., Yu, R. and Bennett, C. F. (20 15) Pharmacokinetics, biodistribution and cell uptake of antisense oligonucleotides. Adv Drug Deliv Rev, 87, 46-51.
6. Gogtay, N.J. and Sridharan, K. (2016) Therapeutic Nucleic Acids: Current clinical status. Br J Clin Pharmacol.
7. Wilds, C. J. and Damha, M. J. (2000) 2′-Deoxy-2′-fluoro-beta-D-arabinonucleosides and oligonucleotides (2′-FANA): synthesis and physicochemical studies. Nucleic Acids Res, 28, 3625-3635.
8. Damha, M. J., Noronha, A. M., Wilds, C. J., Trempe, J. F., Denisov, A., Pon, R. T. and Gehring, K. (2001) Properties of arabinonucleic acids (ANA & 20′F-ANA): implications for the design ofantisense therapeutics that invoke RNase H cleavage of RNA. Nucleosides Nucleotides Nucleic Acids, 20, 429-440.
9. Ferrari, N., Bergeron, D., Tedeschi, A. L., Mangos, M. M., Paquet, L., Renzi, P. M. and Damha, M. J. (2006) Characterization of antisense oligonucleotides comprising 2′-deoxy-2′-fluoro-beta-D-arabinonucleic acid (FANA): specificity, potency, and duration of act ivity. Ann N YAcad Sci, 1082, 91-102
10. Kalota, A., Karabon, L., Swider, C. R., Viazovkina, E., Elzagheid, M., Damha, M. J. and Gewirtz, A. M. (2006) 2′-deoxy-2′-fluoro-beta-D-arabinonucleic acid (2′-FANA) modified oligonucleotides (ON) effect highly efficient, and persistent, gene silencing. Nucleic Acids Res, 34, 45 1-461.
11. Li, F., Sarkhel, S., Wilds, C. J., Wawrzak, Z., Prakash, T. P., Manoharan, M. and Egli, M. (2006) 2′Fluoroarabino- and arabinonucleic acid show different conformations, resulting in deviating RNA affinities and processing of their heteroduplexes with RNA by RNase H. Biochemistry, 45, 4141-4 152.
12. Watts, J. K., Martin-Pintado, N., Gomez-Pinto, I., Schwartzentruber, J., Portella, G., Orozco, M., Gonzalez, C. and Damha, M. J. (20 10) Differential stability of 2′-FANA*RNA and ANA*RNA hybrid duplexes: roles of structure, pseudohydrogen bonding, hydration, ion uptake and flexibility. Nucleic Acids Res, 38, 2498-25 11.
13. Martin-Pintado, N., Yahyaee-Anzahaee, M., Campos-Olivas, R., Noronha, A. M., Wilds, C. J., Damha, M. J. and Gonzalez, C. (201 2) The solution structure of double helical arabino nucleic acids (ANA and 2′-FANA): effect of arabinoses in duplex-hairpin interconversion. Nucleic Acids Res, 40, 9329-9339.
14. Souleimanian, N., Deleavey, G. F., Soifer, H., Wang, S., Tiemann, K., Damha, M. J. and Stei n, C. A. (2012) Antisense 2′-Deoxy, 2′-Fluroarabino Nucleic Acids (2′-FANAs) Oligonucleotides: In Vitro Gymnotic Silencers of Gene Expression Whose Potency Is Enhanced by Fatty Acids. Mol Ther Nucleic Acids, I, e43.
15. Skripkin, E., Paillart, J. C., Marquet, R., Ehresmann, B. and Ehresmann, C. (1994) Identification of the primary site of the human immunodeficiency virus type 1 RNA dimerization in vitro. Proc Natl Acad Sci USA, 91, 4945-4949.
16. Laughrea, M. and Jette, L. (1994) A 19-nucleotide sequence upstream of the 5′ major splice donor is part of the dimerization domain of human immunodeficiency virus I genomic RNA. Biochemistry, 33, 13464-13474.
17. Lu, K., Heng, X., Garyu, L., Monti, S., Garcia, E. L., Kharytonchyk, S., Doljsuren, B., Kulandaivel, G., Jones, S., Hiremath, A., Divakaruni, S. S., LaCotti, C., Barton, S., Tummillo, D., Hosie, A., Edme, K., Albrecht, S., Telesnitsky, A. and Summers, M. F. (2011) NMR detection of structures in the HIV-I 5′-leader RNA that regulate genome packaging. Science, 334, 242-245.
18. Laughrea, M., Jette, L., Mak, J., Kleiman, L., Liang, C. and Wainberg, M. A. (1997) Mutations in the kissing loop hairpin of human immunodeficiency virus type I reduce viral infectivity as well as genomic RNA packaging and dimerization. J Virol, 71, 3397-3406.
19. Elmen, J., Zhang, H. Y., Zuber, B., Ljungberg, K., Wahren, B., Wahlestedt, C. and Liang, Z. (2004) Locked nucleic acid containing antisense oligonucleotides enhance inhibition of HIV-1 genome dimerization and inhibit virus replication. FEBS Lett, 578, 285-290.
20. Lebars, I., Richard, T., Di Primo, C. and Toulme, J. J. (2007) LNA derivatives of a kissing aptamer targeted to the trans-activating responsive RNA element of HIV-1. Blood Cells Mol Dis, 38, 204-209.
21. Parkash, B., Ranjan, A., Tiwari, V., Gupta, S. K., Kaur, N. and Tandon, V. (20 12) Inhibition of5′-UTR RNA conformational switching in HIV-I using antisense PNAs. PloS One, 7, e49310.
22. Reyes-Darias, J. A., Sanchez-Luque, F. J. and Berzal-Herranz, A. (2012) HIV RNA dimerisation interference by antisense oligonucleotides targeted to the 5′ UTR structural elements. Virus Res, 169, 63-71.

1. A method of preventing human immunodeficiency virus (HIV) infection comprising providing a synthetic oligonucleotide comprising a 2′-deoxy-2′-fluoroarabinonucleotide (2′-FANA)-modified nucleotide sequence having a nucleotide gap sequence consists of about 6 to 9 unmodified nucleotides, wherein at least a portion of the synthetic oligonucleotide binds at least a portion of a dimerization initiation site (DIS) of the HIV genomic ribonucleic acid (RNA) molecule, and wherein the synthetic nucleotide prevents HIV infection.

2. The method of claim 1, wherein preventing HIV infection further comprises preventing propagation of HIV in a subject having HIV.

3. The method of claim 2, wherein preventing propagation of HIV in the subject further comprises reducing HIV load in the subject.

4. The method of claim 1, wherein HIV infection is prevented in a cell.

5. The method of claim 1, wherein preventing HIV infection further comprises preventing transmission of HIV from an infected cell to an uninfected cell.

6. The method of claim 1, wherein providing further comprises contacting a cell with the synthetic nucleotide.

7. The method of claim 1, wherein the 2′-FANA-modified nucleotide sequence binds at least the portion of the DIS of the HIV genomic RNA molecule with full complementarity or partial complementarity, and wherein inhibiting dimerization of the HIV genome is induced by RNase H activity, steric hindrance, or a combination thereof.

8. The method of claim 1, wherein the synthetic oligonucleotide comprises at least nine successive nucleotides of SEQ ID NO: 1 or a sequence complementary to at least nine successive nucleotides of SEQ ID NO: 1.

9. The method of claim 1, wherein the synthetic oligonucleotide comprises a nucleotide sequence of SEQ ID NO: 10, or SEQ ID NO: 11.

10. The method of claim 1, wherein internucleotide linkages between nucleotides of the synthetic oligonucleotide are phosphodiester bonds, phosphotriester bonds, phosphorothioate bonds, phosphorodithioate bonds, Rp-phosphorothioate bonds, Sp-phosphorothioate bonds, boranophosphate bonds, methylene bonds(methylimino), amide bonds, methylphosphonate bonds, 3′-thioformacetal bonds, amide bonds, phosphoramidate groups, or any combination thereof.

11. The method of claim 1, wherein the synthetic oligonucleotide comprises 9 unmodified nucleotides.

12. The method of claim 1, wherein the synthetic oligonucleotide has a formula set forth below:
(i) when the synthetic oligonucleotide is 12 nucleotides in length, it has a formula of [symbol]
XXXXXX [symbol] ;
(ii) when the synthetic oligonucleotide is 14 nucleotides in length, it has a formula of [symbol]
XXXXXX [symbol] ;
(iii) when the synthetic oligonucleotide is 17 nucleotides in length, it has a formula of [symbol]
XXXXXXXXX [symbol] ;
(iv) when the synthetic oligonucleotide is 18 nucleotides in length, it has a formula selected from [symbol] XXXXXXXX [symbol] , and [symbol] XXXXXXX [symbol] ;
(v) when the synthetic oligonucleotide is 20 nucleotides in length, it has a formula of selected from [symbol] XXXXXXXXX [symbol] , and [symbol] XXXXXXXX [symbol] ; or
(vi) when the synthetic oligonucleotide is 21 nucleotides in length, it has a formula of [symbol]
XXXXXXXXX [symbol] ,
wherein X represents a nucleotide selected from the group consisting of A, C, G, T and U, and wherein the bold and italicized nucleotides represent sugar-modified or 2′-FANA-modified nucleotides.

13. The method of claim 1, wherein delivery of the synthetic oligonucleotide is via gymnotic delivery.

14. A method of preventing propagation of HIV from an HIV infected cell into an HIV uninfected cell comprising delivering a synthetic oligonucleotide comprising a 2′-deoxy-2′-fluoroarabinonucleotide (2′-FANA)-modified nucleotide sequence having a nucleotide gap sequence consists of about 6 to 9 unmodified nucleotides, wherein at least a portion of the synthetic oligonucleotide binds at least a portion of a dimerization initiation site (DIS) of the HIV genomic ribonucleic acid (RNA) molecule, and wherein the synthetic nucleotide prevents HIV infection.

15. The method of claim 14, wherein the 2′-FANA-modified nucleotide sequence binds at least the portion of the DIS of the HIV genomic RNA molecule with full complementarity or partial complementarity, and wherein inhibiting dimerization of the HIV genome is induced by RNase H activity, steric hindrance, or a combination thereof.

16. The method of claim 14, wherein the synthetic oligonucleotide comprises at least nine successive nucleotides of SEQ ID NO: 1 or a sequence complementary to at least nine successive nucleotides of SEQ ID NO: 1.

17. The method of claim 14, wherein the synthetic oligonucleotide comprises a nucleotide sequence of SEQ ID NO: 10, or SEQ ID NO: 11.

18. The method of claim 14, wherein internucleotide linkages between nucleotides of the synthetic oligonucleotide are phosphodiester bonds, phosphotriester bonds, phosphorothioate bonds, phosphorodithioate bonds, Rp-phosphorothioate bonds, Sp-phosphorothioate bonds, boranophosphate bonds, methylene bonds(methylimino), amide bonds, methylphosphonate bonds, 3′-thioformacetal bonds, amide bonds, phosphoramidate groups, or any combination thereof.

19. The method of claim 14, wherein the synthetic oligonucleotide has a formula set forth below:
(i) when the synthetic oligonucleotide is 12 nucleotides in length, it has a formula of [symbol]
XXXXXX [symbol] ;
(ii) when the synthetic oligonucleotide is 14 nucleotides in length, it has a formula of [symbol]
XXXXXX [symbol] ;
(iii) when the synthetic oligonucleotide is 17 nucleotides in length, it has a formula of [symbol]
XXXXXXXXX [symbol] ;
(iv) when the synthetic oligonucleotide is 18 nucleotides in length, it has a formula selected from [symbol] XXXXXXXX [symbol] , and [symbol] XXXXXXX [symbol] ;
(v) when the synthetic oligonucleotide is 20 nucleotides in length, it has a formula of selected from [symbol] XXXXXXXXX [symbol] , and [symbol] XXXXXXXX [symbol] X; or
(vi) when the synthetic oligonucleotide is 21 nucleotides in length, it has a formula of [symbol]
XXXXXXXXX [symbol] ,
wherein X represents a nucleotide selected from the group consisting of A, C, G, T and U, and wherein the bold and italicized nucleotides represent sugar-modified or 2′-FANA-modified nucleotides.

20. The method of claim 14, wherein delivery of the synthetic oligonucleotide is via gymnotic delivery.


SEQUENCE LISTING (54 sequences)

SEQ ID NO: 1 - DNA, 35 bp, synthetic construct
  misc_feature 1..35: note = HIV dimerization initiation site
  source 1..35: mol_type = other DNA
gacggcttgctgaagcgcgcacggcaagaggcgtc

SEQ ID NO: 2-4 - skipped (3 entries)

SEQ ID NO: 5 - DNA, 18 bp, synthetic construct
  misc_feature 1..18: note = antisense oligonucleotide
  source 1..18: mol_type = other DNA
gccgtgtgcacttcagca

SEQ ID NO: 6 - DNA, 18 bp, synthetic construct
  misc_feature 1..18: note = antisense oligonucleotide
  source 1..18: mol_type = other DNA
gccgtgtgcacttcagca

SEQ ID NO: 7 - RNA, 20 nt, synthetic construct
  misc_feature 1..20: note = antisense oligonucleotide
  source 1..20: mol_type = other RNA
ttgccgtgtgcacttcagaa

SEQ ID NO: 8 - DNA, 20 bp, synthetic construct
  misc_feature 1..20: note = antisense oligonucleotide
  source 1..20: mol_type = other DNA
ttgccgtgtgcacttcagca

SEQ ID NO: 9 - DNA, 20 bp, synthetic construct
  misc_feature 1..20: note = antisense oligonucleotide
  source 1..20: mol_type = other DNA
ttgccgtgtgcacttcagca

SEQ ID NO: 10 - DNA, 20 bp, synthetic construct
  misc_feature 1..20: note = antisense oligonucleotide
  source 1..20: mol_type = other DNA
tgccgtgtgcacttcagcaa

SEQ ID NO: 11 - DNA, 20 bp, synthetic construct
  misc_feature 1..20: note = antisense oligonucleotide
  source 1..20: mol_type = other DNA
tgccgtgtgcacttcagcaa

SEQ ID NO: 12 - DNA, 19 bp, synthetic construct
  misc_feature 1..19: note = antisense oligonucleotide
  source 1..19: mol_type = other DNA
tgccgtgtgcacttcagca

SEQ ID NO: 13 - DNA, 21 bp, synthetic construct
  misc_feature 1..21: note = antisense oligonucleotide
  source 1..21: mol_type = other DNA
ttgccgtgtgcacttcagcaa

SEQ ID NO: 14 - DNA, 21 bp, synthetic construct
  misc_feature 1..21: note = antisense oligonucleotide
  source 1..21: mol_type = other DNA
ttgccgtgtgcacttcagcaa

SEQ ID NO: 15 - DNA, 21 bp, synthetic construct
  misc_feature 1..21: note = antisense oligonucleotide
  source 1..21: mol_type = other DNA
ttgccgtgtgcacttcagcaa

SEQ ID NO: 16 - DNA, 14 bp, synthetic construct
  misc_feature 1..14: note = antisense oligonucleotide
  source 1..14: mol_type = other DNA
ctcgcctcttgccg

SEQ ID NO: 17 - DNA, 14 bp, synthetic construct
  misc_feature 1..14: note = antisense oligonucleotide
  source 1..14: mol_type = other DNA
ctcgcctcttgccg

SEQ ID NO: 18 - DNA, 14 bp, synthetic construct
  misc_feature 1..14: note = antisense oligonucleotide
  source 1..14: mol_type = other DNA
ctcgcctcttgccg

SEQ ID NO: 19 - RNA, 14 nt, synthetic construct
  misc_feature 1..14: note = antisense oligonucleotide
  source 1..14: mol_type = other RNA
ctcgcctcttgccg

SEQ ID NO: 20 - DNA, 14 bp, synthetic construct
  misc_feature 1..14: note = antisense oligonucleotide
  source 1..14: mol_type = other DNA
ctcgcctcttgccg

SEQ ID NO: 21 - DNA, 12 bp, synthetic construct
  misc_feature 1..12: note = antisense oligonucleotide
  source 1..12: mol_type = other DNA
cagcaagccgag

SEQ ID NO: 22 - DNA, 21 bp, synthetic construct
  misc_feature 1..21: note = antisense oligonucleotide
  source 1..21: mol_type = other DNA
cgcctcttgccgtgtgcactt

SEQ ID NO: 23 - DNA, 20 bp, synthetic construct
  misc_feature 1..20: note = antisense oligonucleotide
  source 1..20: mol_type = other DNA
gcctcttgccgtgtgcactt

SEQ ID NO: 24 - DNA, 21 bp, synthetic construct
  misc_feature 1..21: note = antisense oligonucleotide
  source 1..21: mol_type = other DNA
cgcctcttgccgtgtgcactt

SEQ ID NO: 25 - DNA, 21 bp, synthetic construct
  misc_feature 1..21: note = antisense oligonucleotide
  source 1..21: mol_type = other DNA
cgcctcttgccgtgtgcactt

SEQ ID NO: 26 - DNA, 21 bp, synthetic construct
  misc_feature 1..21: note = antisense oligonucleotide
  source 1..21: mol_type = other DNA
cgcctcttgccgtgtgcactt

SEQ ID NO: 27 - DNA, 19 bp, synthetic construct
  misc_feature 1..19: note = antisense oligonucleotide
  source 1..19: mol_type = other DNA
ctcttgccgtgtgcacttc

SEQ ID NO: 28 - DNA, 18 bp, synthetic construct
  misc_feature 1..18: note = antisense oligonucleotide
  source 1..18: mol_type = other DNA
ctcttgccgtgtgcactt

SEQ ID NO: 29 - DNA, 18 bp, synthetic construct
  misc_feature 1..18: note = antisense oligonucleotide
  source 1..18: mol_type = other DNA
ctcttgccgtgtgcactt

SEQ ID NO: 30 - DNA, 18 bp, synthetic construct
  misc_feature 1..18: note = antisense oligonucleotide
  source 1..18: mol_type = other DNA
ctcttgccgtgtgcactt

SEQ ID NO: 31 - RNA, 20 nt, synthetic construct
  misc_feature 1..20: note = antisense oligonucleotide
  source 1..20: mol_type = other RNA
tgtgcacttcagcaagccga

SEQ ID NO: 32 - DNA, 19 bp, synthetic construct
  misc_feature 1..19: note = antisense oligonucleotide
  source 1..19: mol_type = other DNA
gtgtgcacttcagcaagcc

SEQ ID NO: 33 - DNA, 19 bp, synthetic construct
  misc_feature 1..19: note = antisense oligonucleotide
  source 1..19: mol_type = other DNA
gtgtgcacttcagcaagcc

SEQ ID NO: 34 - DNA, 18 bp, synthetic construct
  misc_feature 1..18: note = antisense oligonucleotide
  source 1..18: mol_type = other DNA
tgtgcacttcagcaagcc

SEQ ID NO: 35 - DNA, 21 bp, synthetic construct
  misc_feature 1..21: note = antisense oligonucleotide
  source 1..21: mol_type = other DNA
tgagctcttcgtcgctgtctc

SEQ ID NO: 36 - DNA, 20 bp, synthetic construct
  misc_feature 1..20: note = antisense oligonucleotide
  source 1..20: mol_type = other DNA
tgagctcttcgtcgctgtct

SEQ ID NO: 37 - DNA, 21 bp, synthetic construct
  misc_feature 1..21: note = antisense oligonucleotide
  source 1..21: mol_type = other DNA
gtctgagggatctctagttac

SEQ ID NO: 38 - DNA, 20 bp, synthetic construct
  misc_feature 1..20: note = antisense oligonucleotide
  source 1..20: mol_type = other DNA
tctgagggatctctagttac

SEQ ID NO: 39 - DNA, 22 bp, synthetic construct
  misc_feature 1..22: note = antisense oligonucleotide
  source 1..22: mol_type = other DNA
gtgagctcttcgtcgctgtctc

SEQ ID NO: 40 - DNA, 15 bp, synthetic construct
  misc_feature 1..15: note = antisense oligonucleotide
  source 1..15: mol_type = other DNA
tgagctcttcgtcgc

SEQ ID NO: 41 - DNA, 16 bp, synthetic construct
  misc_feature 1..16: note = antisense oligonucleotide
  source 1..16: mol_type = other DNA
ctgagggtctctagtt

SEQ ID NO: 42 - DNA, 21 bp, synthetic construct
  misc_feature 1..21: note = antisense oligonucleotide
  source 1..21: mol_type = other DNA
gtctgagggatctctagttac

SEQ ID NO: 43 - skipped

SEQ ID NO: 44 - DNA, 26 bp, synthetic construct
  misc_feature 1..26: note = antisense oligonucleotide
  source 1..26: mol_type = other DNA
cttgccgtgtgcacttcagcaagccg

SEQ ID NO: 45 - DNA, 14 bp, synthetic construct
  misc_feature 1..14: note = antisense oligonucleotide
  source 1..14: mol_type = other DNA
ctcgcctcttgccg

SEQ ID NO: 46 - DNA, 23 bp, synthetic construct
  misc_feature 1..23: note = antisense oligonucleotide
  source 1..23: mol_type = other DNA
ctcgcctcttgccgtgtgcactt

SEQ ID NO: 47 - DNA, 21 bp, synthetic construct
  misc_feature 1..21: note = antisense oligonucleotide
  source 1..21: mol_type = other DNA
tgtgcacttcagcaagccgag

SEQ ID NO: 48 - RNA, 20 nt, synthetic construct
  misc_feature 1..20: note = antisense oligonucleotide
  source 1..20: mol_type = other RNA
tgccgtgtgcacttcagcaa

SEQ ID NO: 49 - DNA, 20 bp, synthetic construct
  misc_feature 1..20: note = antisense oligonucleotide
  source 1..20: mol_type = other DNA
tgccgtgtgcacttcagcaa

SEQ ID NO: 50 - RNA, 21 nt, synthetic construct
  misc_feature 1..21: note = antisense oligonucleotide
  source 1..21: mol_type = other RNA
ttgccgtgtgcacttcagcaa

SEQ ID NO: 51 - RNA, 21 nt, synthetic construct
  misc_feature 1..21: note = antisense oligonucleotide
  source 1..21: mol_type = other RNA
tgtgcacttcagcaagccgag

SEQ ID NO: 52 - DNA, 17 bp, synthetic construct
  misc_feature 1..17: note = antisense oligonucleotide
  source 1..17: mol_type = other DNA
ctgagggatctctagtt

SEQ ID NO: 53 - DNA, 18 bp, synthetic construct
  misc_feature 1..18: note = antisense oligonucleotide
  source 1..18: mol_type = other DNA
tgtgcacttcagcaagcc

SEQ ID NO: 54 - DNA, 18 bp, synthetic construct
  misc_feature 1..18: note = antisense oligonucleotide
  source 1..18: mol_type = other DNA
tgtgcacttcagcaagcc